SEQUENCE LISTING (3 sequences)

SEQ ID NO: 1 - RNA, 10 nt, Artificial Sequence
  totally synthetic
aaaaaaaagu

SEQ ID NO: 2 - RNA, 10 nt, Artificial Sequence
  totally synthetic
aaaaaaaagc

SEQ ID NO: 3 - RNA, 10 nt, Artificial Sequence
  totally synthetic
aaaaaaaacc

(19) United States
(12) Patent Application Publication
(10) Pub. No.: US 2006/0074035 A1
(43) Pub. Date: Apr. 6, 2006

(54) Dinucleotide inhibitors of de novo RNA polymerases for treatment or prevention of viral infections

(76) Inventors: Zhi Hong, Aliso Viejo, CA (US); Weidong Zhong, Laguna Niguel, CA (US); Haoyun An, Carlsbad, CA (US); Dinesh Barawkar, Foothill Ranch, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/418,662

(22) Filed: Apr. 17, 2003

Related U.S. Application Data
(60) Provisional application No. 60/373,735, filed on Apr. 17, 2002.

(51) Int. Cl. A61K 31/7076; C07H 19/04

(52) U.S. Cl. 514/44; 536/26.1

(57) ABSTRACT

Contemplated dinucleotide compounds have a general structure of A-B and inhibit synthesis of an RNA-dependent polymerase that initiates RNA replication de novo. In preferred dinucleotides, A comprises a purine or modified purine heterocyclic base and B comprises a pyrimidine or modified pyrimidine heterocyclic base.

This application claims the benefit of U.S. provisional patent with the Ser. No. 60/373,735, which was filed Apr. 17, 2002, and which is incorporated by reference herein.

### FIELD OF THE INVENTION

The present invention relates to the synthesis and utilization of dinucleotide analogues as inhibitors of viral RNA polymerases that use a de novo mechanism for initiation of RNA replication.

### BACKGROUND OF THE INVENTION

HCV infection poses a significant and worldwide public health problem and is generally recognized as the major cause of non-A, non-B hepatitis. Although HCV infection resolves in some cases, the virus establishes chronic infection in up to 80% of the infected individuals persisting for decades. It is estimated that about 20% of these infected individuals will go on to develop cirrhosis and 1 to 5% will develop liver failure and hepatocellular carcinoma (Seeff, et al. 1999, Am. J. Med. 107:10S-15S; Saito, et al. 1990, Proc. Natl. Acad. Sci. USA, 87:6547-6549; WHO, 1996, Weekly Epidemiol. Res, 71:346-349). Chronic hepatitis C is the leading cause of chronic liver disease and the leading indication for liver transplantation in the United States of America. The Centers for Disease Control and Prevention estimate that hepatitis C currently is responsible for approximately 8,000 to 10,000 deaths in the United States annually. This number is projected to increase significantly over the next decade. Currently, there is no vaccine for HCV infection due to the high degree of heterogeneity of this virus and high immune evasion.

The objectives for the treatment of chronic hepatitis C are to achieve a complete and sustained clearance of HCV RNA in serum and normalization of serum alanine aminotransferase (ALT) levels. The current treatment options for chronic hepatitis C include (pegalated) IFN-α monotherapy and (pegalated) IFN-α and ribavirin combination therapy, with sustained virological response rates between 10% and 60%. Clearly, more effective and more direct antiviral interventions are necessary for further prevention and treatment of the life threatening complications caused by HCV infection.

HCV is a positive-strand RNA virus belonging to the Flaviviridae family (Choo, et al., 1989, Science 244:359-362). This virus family also contains more than 38 flaviviruses that are associated with human diseases, including the dengue fever viruses, yellow fever viruses and Japanese encephalititis virus, and pestiviruses whose infection of domesticated livestock can cause significant economic losses worldwide. Like other RNA viruses and virally encoded replication enzymes, RNA-dependent RNA polymerase (RdRp) plays a central role in viral RNA replication of HCV and other members of the Flaviviridae family. In the case of HCV, the replication protein is termed NS5B (nonstructural protein 5B). RdRp proteins are the key components of the viral replicase complexes and therefore serve as the attractive targets for antiviral development.

The RNA replication is thought to be initiated by HCV NS5B via a de novo or primer-independent mechanism. As initiation process has been considered the rate-limiting step in viral RNA replication, inhibitors that interfere with the initiation process appear to be promising candidates for suppression of virus replication. Consequently, inhibitors of the de novo replication may provide a significant tool in the treatment and/or prevention of viral infections with viruses that use a de novo mechanism for initiation of RNA replication. Therefore, there is still a need to provide antiviral drugs and methods, especially as they relate to inhibition of the de novo replication of a RdRp of a virus.

### SUMMARY OF THE INVENTION

The present invention is directed to compositions and methods in which a dinucleotide analog inhibits de novo viral replication by inhibition of the RdRp. It is generally preferred that a compound comprises a dinucleotide of the structure A-B, wherein A and B independently comprise a nucleoside, a nucleoside analog, a nucleotide, or a nucleotide analog.

In one aspect of the inventive subject matter, A comprises a purine nucleoside or purine nucleoside analog, and/or B comprises a pyrimidine nucleoside or pyrimidine nucleoside analog, and it is still further preferred that the sugar moiety of A is covalently bound to B, which may include coupling of A to B via a group comprising a phosphorous atom (e.g., a phosphate, a phosphorothioate, a phosphonate, a phosphonamide, or a boranophosphate). Additionally, or alternatively, at least one of A and B may include a modified sugar (e.g., modified to include a 2′-methyl or methoxy group or a 3′-methyl or methoxy group).

In still further contemplated aspects, at least one of A and B comprises guanosine, and/or at least one of A and B includes a heterocyclic base that forms at least two hydrogen bonds with a terminal heterocyclic base of a template RNA. While not limiting to the inventive concept presented herein, it is typically preferred that the de novo initiation of RNA replication is initiated by guanosine triphosphate, adenosine triphosphate, or a dinucleotide. Therefore, a particularly preferred RNA polymerases is the HCV NS5B polypeptide.

In a further aspect of the inventive subject matter, the dinucleotide will have a structure according to Formulae 1, 2, or 3, wherein the substituents are defined as in the section entitled “Contemplated Compounds” below.

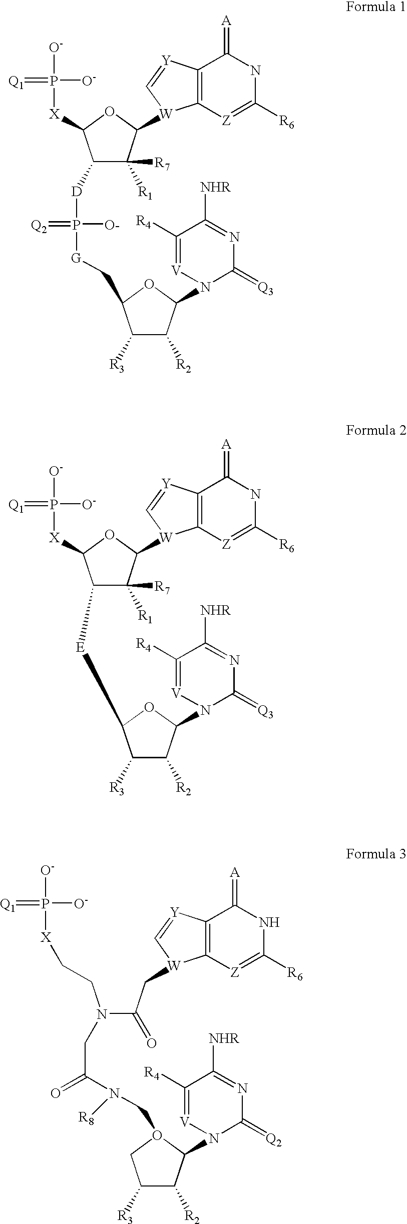

### BRIEF DESCRIPTION OF THE DRAWING

FIG. 1 is an autoradiograph depicting inhibition of GTP-initiated RNA synthesis by exemplary dinucleotides according to the inventive subject matter.

FIG. 2 is an autoradiograph depicting inhibition of ATP-initiated RNA synthesis by an exemplary dinucleotide according to the inventive subject matter.

FIG. 3 is an autoradiograph depicting inhibition of GpC-primed RNA synthesis by exemplary dinucleotides according to the inventive subject matter.

FIG. 4 is an autoradiograph depicting lack of inhibition of GG-primed RNA synthesis by exemplary dinucleotides according to the inventive subject matter.

FIG. 5 is an exemplary scheme for synthesis of selected dinucleotides according to the inventive subject matter.

FIG. 6 is an exemplary scheme for synthesis of further dinucleotides according to the inventive subject matter.

FIG. 7 is an exemplary scheme for synthesis of a selected 5′-methylene phosphonate nucleoside according to the inventive subject matter.

FIG. 8 is an exemplary scheme for synthesis of selected phosphoramidates according to the inventive subject matter.

FIG. 9 is an exemplary scheme for synthesis of a selected 5′-methylene phosphonate dinucleotide analog according to the inventive subject matter.

### DETAILED DESCRIPTION OF THE INVENTION

The inventors discovered that a compound comprising a dinucleotide of the structure A-B inhibits synthesis of a RNA de novo polymerase, wherein A and/or B are or include a nucleoside, nucleoside analog, a nucleotide, or nucleotide analog. Particularly contemplated compounds may be useful for treatment or prevention of Flaviviridae viral infections, and especially contemplated viruses of the Flaviviridae family include bovine viral diarrhea virus (BVDV), yellow fever viruses, and particularly hepatitis C virus (HCV).

The term “dinucleotide” as used herein refers to a compound in which two nucleosides, nucleotides, or analogs thereof are coupled together to form a single molecule. Particularly preferred coupling between the nucleosides include covalent coupling by linker groups (e.g., phosphonate, phosphate, phosphorothioate, boranophosphate, amide, etc.), and it is still further preferred that an OH group or a group other than an OH group is covalently bound to the C5′-atom of the nucleoside locate at the 5′-end of the dinucleotide. The term “nucleoside” as used herein refers to all compounds in which a heterocyclic base is covalently coupled to a sugar, and an especially preferred coupling of the nucleoside to the sugar includes a C1′-(glycosidic) bond of a carbon atom in a sugar to a carbon- or heteroatom (typically nitrogen) in the heterocyclic base. The term “nucleoside analog” as used herein refers to all nucleosides in which the sugar is not a ribofuranose and/or in which the heterocyclic base is not a naturally occurring base (e.g., A, G, C, T, I, etc.), however, also includes nucleosides. The term “nucleotide” as used herein refers to a nucleoside that is covalently bound via the sugar (preferably at the C5′-position) to a modified or unmodified phosphate or phosphonate group. Similarly, the term “nucleotide analog” refers to all nucleotides in which the sugar is not a ribofuranose and/or in which the heterocyclic base is not a naturally occurring base (e.g., A, G, C, T, I, etc.), however, also includes nucleotides.

Also, as used herein, the term “heterocycle” refers to any compound in which a plurality of atoms form a ring via a plurality of covalent bonds, wherein the ring includes at least one atom other than a carbon atom. Particularly contemplated heterocycles include 5- and 6-membered rings with nitrogen, sulfur, or oxygen as the non-carbon atom (e.g., imidazole, pyrrole, triazole, dihydropyrimidine).

As further used herein, the term “sugar” refers to all carbohydrates and derivatives thereof, wherein particularly contemplated derivatives include deletion, substitution or addition of a chemical group in the sugar. For example, especially contemplated deletions include 2′-deoxy and/or 3′-deoxy sugars. Especially contemplated substitutions include replacement of the ring-oxygen with sulfur, methylene, or nitrogen, or replacement of a hydroxyl group with a halogen, an amino-, sulfhydryl-, or methyl group, and especially contemplated additions include methylene phosphonate groups, and 2′ and/or 3′-methyl and/or methoxy groups (which may be in alpha or beta orientation). Further contemplated sugars also include sugar analogs (i.e., not naturally occurring sugars), and particularly carbocyclic ring systems.

The terms “alkyl” and “unsubstituted alkyl” are used interchangeably herein and refer to any linear, branched, or cyclic hydrocarbon in which all carbon-carbon bonds are single bonds. The term “substituted alkyl” as used herein refers to any alkyl that further comprises a functional group, and particularly contemplated functional groups include nucleophilic (e.g., —NH₂, —OH, —SH, —NC, etc.) and electrophilic groups (e.g., C(O)OR, C(X)OH, etc.), polar groups (e.g., —OH), non-polar groups (e.g., aryl, alkyl, alkenyl, alkynyl, etc.), ionic groups (e.g., —NH₃⁺), halogens (e.g., —F, —Cl), and all chemically reasonable combinations thereof. The terms “alkenyl” and “unsubstituted alkenyl” are used interchangeably herein and refer to any linear, branched, or cyclic alkyl with at least one carbon-carbon double bond. The term “substituted alkenyl” as used herein refers to any alkenyl that further comprises a functional group, and particularly contemplated functional groups include those discussed above.

Furthermore, the terms “alkynyl” and “unsubstituted alkynyl” are used interchangeably herein and refer to any linear, branched, or cyclic alkyl or alkenyl with at least one carbon-carbon triple bond. The term “substituted alkynyl” as used herein refers to any alkynyl that further comprises a functional group, and particularly contemplated functional groups include those discussed above. The terms “aryl” and “unsubstituted aryl” are used interchangeably herein and refer to any aromatic cyclic alkenyl or alkynyl. The term “substituted aryl” as used herein refers to any aryl that further comprises a functional group, and particularly contemplated functional groups include those discussed above. The term “alkaryl” is employed where the aryl is further covalently bound to an alkyl, alkenyl, or alkynyl. It should still further be appreciated that each of the contemplated alkyls, alkenyls, alkynyls, aryls, alkaryls, and heterocycles may independently and optionally be substituted to yield the corresponding substituted alkyls, alkenyls, alkynyls, aryls, alkaryls, and heterocycles.

Thus, the term “substituted” as used herein also refers to a replacement of a chemical group or substituent (typically H or OH) with a functional group (i.e., a group other than H or OH), and particularly contemplated functional groups include nucleophilic (e.g., —NH₂, —OH, —SH, —NC, etc.) and electrophilic groups (e.g., C(O)OR, C(X)OH, etc.), polar groups (e.g., —OH), non-polar groups (e.g., aryl, alkyl, alkenyl, alkynyl, etc.), ionic groups (e.g., —NH₃⁺), halogens (e.g., —F, —Cl), and all chemically reasonable combinations thereof.

As still further used herein, the term “inhibits synthesis of a polymerase” refers to a partial, or even complete inhibition of the catalytic activity of the polymerase, wherein the catalytic activity includes initiation (i.e., formation of a dinucleotide in the presence of a template) as well as elongation of a di-, oligo-, or polynucleotide. Inhibition may be due to one or more factors, and especially contemplated modes of inhibition include competitive inhibition, allosteric inhibition, and non-competitive inhibition. Therefore, inhibition of synthesis especially includes partial or even reduction of catalytic activity. As also used herein, the term “a polymerase that initiates RNA replication de novo” refers to a polymerase that initiates RNA synthesis in the presence of an RNA template without a oligonucleotide primer (which may or may not be provided by the template).

Contemplated Compounds

Contemplated compounds will generally have a structure of A-B, wherein A and B represent a nucleoside or nucleotide (or analog thereof), and wherein A and B are coupled together to form the dinucleotide compound. While not limiting the inventive subject matter, it is generally preferred that A will comprise a purine-type heterocyclic base (i.e., a 5-membered ring fused to a 6-membered ring with at least one nitrogen heteroatom), while B will comprise a pyrimidine-type heterocyclic base (i.e., a 6-membered ring with at least one nitrogen heteroatom).

Depending on the particular RNA polymerase and on the nucleotide sequence at the initiation site, it is preferred that A comprises a purine nucleoside or purine nucleoside analog and B comprises a pyrimidine nucleoside or pyrimidine nucleoside analog. Such dinucleotides are considered particularly useful as inhibitors of an HCV NS5B polypeptide in conjunction with the HCV RNA as a template.

Further particularly preferred compounds may include a phosphate, phosphonate, or thiophosphate/thiophosphonate group as a covalent bond between A and B, and especially preferred compounds are depicted below in Formulae 1-3. Thus, in one aspect of the inventive subject matter, a dinucleotide according to the inventive subject matter may have a structure according to Formula 1

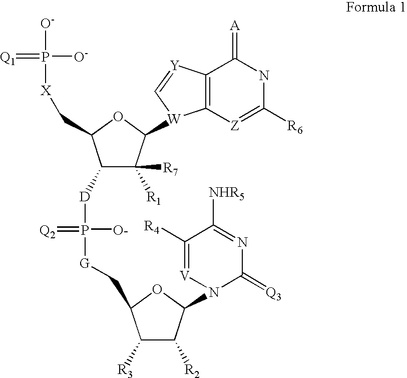

wherein Q₁, Q₂, and Q₃ are independently O or S; X is O, S, NH, NR, CH₂, CF₂, CHR, or a bond between the C5′-carbon and the P atom; Y is CH, COR′, or N, wherein R′ is R, CN, COOH, COOR, CONHR, or C(═NH)NH₂; V and Z are independently N, CH, or CR; W is N or C; A is O, S, NH, or NR; D is O, S, NH, or CH₂; G is O, S, NH, CH₂, CF₂, or a bond between the C5′-carbon and the P atom; R₁, R₂, R₃ are independently H, OH, OR, R, halo, CF₃, CCl₃, CHCl₂, CH₂OH, NO₂, CN, N₃, SH, SR, NH₂, NHR, NHCOR, NHSO₂R, NHCONHR, NHCSNHR; R₄ is R, halogen, haloalkyl, haloalkenyl, or substituted heteroaryl; R₅ is H, NH₂, NHR, NR₂, NHCOR, NHSO₂R, heterocycle, COR, or SO₂R; R₆ is NH₂, NHCOR, NHSO₂R, NHNH₂, NHNHR, NHR, or NR₂; R₇ is H, OH, SH, OR, SR, R, halogen, CF₃, CN, CHCl₂, CH₂OH, N₃, NH₂, or CH₂Cl; and wherein R is alkyl, alkenyl, alkynyl, aryl, or alkaryl.

In another aspect of the inventive subject matter, a dinucleotide according to the inventive subject matter may have a structure according to Formula 2

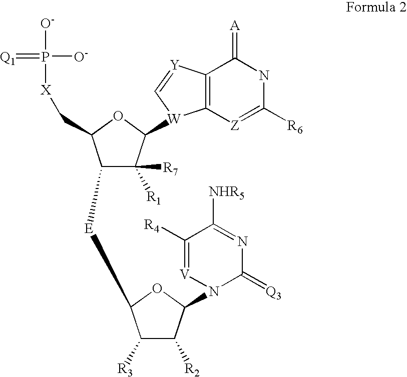

wherein E is —O—P(Q₂)(NR₈R₉)—O—, —NHC(O)(CH₂)₁₋₁₀C(O)—, —NH-Heterocycle-O—, —O(CH₂)₁₋₁₀C(O)—, or —O-Heterocycle-O—; wherein Q₁, Q₂, and Q₃ are independently O or S; X is O, S, NH, NR, CH₂, CF₂, CHR, or a bond between the C5′-carbon and the P atom; Y is CH, COR′, or N, wherein R′ is R, CN, COOH, COOR, CONHR, or C(═NH)NH₂; V and Z are independently N, CH, or CR; W is N or C; A is O, S, NH, or NR; R₁, R₂, R₃ are independently H, OH, OR, R, halo, CF₃, CCl₃, CHCl₂, CH₂OH, NO₂, CN, N₃, SH, SR, NH₂, NHR, NHCOR, NHSO₂R, NHCONHR, NHCSNHR; R₄ is R, halogen, haloalkyl, haloalkenyl, or substituted heteroaryl; R₅ is H, NH₂, NHR, NR₂, NHCOR, NHSO₂R, heterocycle, COR, or SO₂R; R₆ is NH₂, NHCOR, NHSO₂R, NHNH₂, NHNHR, NHR, or NR₂; R₇ is H, OH, SH, OR, SR, R, halogen, CF₃, CN, CHCl₂, CH₂OH, N₃, NH₂, or CH₂Cl; R₈ and R₉ are independently H, (CH₂)₁₋₁₀-NH₂, (CH₂)₁₋₁₀—C(═NR)NHR, (CH₂)₁₋₁₀—NH—C(H₂)₁₋₁₀—COOR, (CH₂)₁₋₁₀—CONHR, (CH₂)₁₋₁₀-Heterocycles, or R; and wherein R is alkyl, alkenyl, alkynyl, aryl, or alkaryl.

In a still further aspect of the inventive subject matter, a dinucleotide according to the inventive subject matter may have a structure according to Formula 3

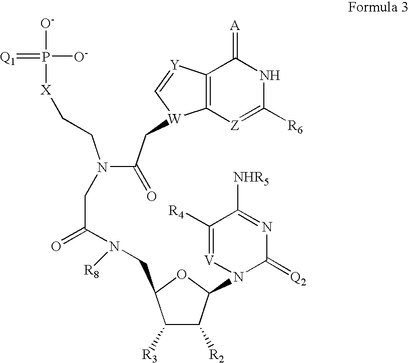

wherein X is O, S, NH, NR, CH₂, a covalent bond between the P atom and the CH₂ group, CF₂, or CHR; Q₁ and Q₂ are independently O or S; Y is CH, COR′, or N, wherein R′ is CN, Me, COOH, COOR, CONHR, C(═NH)NH₂, or R; Z and V are independently N, CH, or CR; W is N or C; A is O, S, NH, or NR; R₂ and R₃ are independently H, OH, OR, R, halo, CF₃, CCl₃, CHCl₂, CH₂OH, NO₂, CN, N₃, SH, SR, NH₂, NHR, NHCOR, NHSO₂R, NHCONHR, NHCSNHR; R₄ is R, halogen, haloalkyl, haloalkenyl, or substituted heteroaryl; R₅ is H, NH₂, NHR, NR₂, NHCOR, NHSO₂R, heterocycle, COR, or SO₂R; R₆ is NH₂, NHCOR, NHSO₂R, NHNH₂, NHNHR, NHR, or NR₂; R₈ is H or R; and wherein R is alkyl, alkenyl, alkynyl, aryl, or alkaryl.

It should further be appreciated that the nature of particular groups in the dinucleotide may vary considerably, and in one set of alternative aspects, the sugar moiety is modified. For example, suitable modifications include replacement of the ring oxygen with a substituted nitrogen (NR), a sulfur atom, or an optionally substituted methylene group. In another example, one or more of the C2′-, C3′-, and C4′-substituents may be replaced with a methyl or methoxy group. In still further examples, the sugar may include a double bond in the ring.

With respect to the coupling of the first nucleoside/nucleotide (or analog thereof) to the second nucleoside/nucleotide (or analog thereof), it is generally contemplated that all covalent couplings are deemed suitable for use herein, and that the coupling may be directly (i.e., one substituent of the first sugar reacts with one substituent of the second sugar) or indirectly (e.g., via a bifunctional linker). However, especially preferred coupling s include those in which A is covalently bound to B via a chemical group that includes a phosphorous atom (e.g., via a phosphate group, a phosphorothioate group, a phosphonate group, a phosphoamidate group, a phosphonamide group, or a boranophosphate group).

Similarly, it is contemplated that the nature of the heterocyclic base in the dinucleotide may vary, and it should be appreciated that the choice of a particular heterocyclic base will at least in part depend on the particular template strand. However, it is generally preferred that at least one of A and B includes a heterocyclic base that forms at least two hydrogen bonds with a terminal heterocyclic base of a template RNA.

In yet further aspects of the inventive subject matter, it should be recognized that the compounds presented herein may also be prepared in form of a prodrug, and all known prodrug forms are deemed suitable for use herein. However, especially preferred prodrugs include those that exhibit increased specificity towards a target cell (e.g., hepatocyte) and/or target organ (e.g., liver), and/or exhibit decreased toxicity against non-target cells and/or a non-target organ. Thus, suitable prodrug forms include modifications that can be specifically removed by the hepatic CYP system. Similarly, it should also be recognized that the compounds presented herein may be converted in vivo to one or more metabolites, wherein the metabolite may have desirable pharmceutical properties (e.g., inhibits the synthesis of a polymerase that initiates RNA replication de novo).

Synthesis of Contemplated Compounds

It should generally be recognized that there are numerous synthetic procedures that may be employed to generate contemplated compounds from nucleosides, nucleotides, and analogs thereof, and all of the known methods are deemed suitable for use in conjunction with the teachings presented herein. For example, suitable methods include classic solvent synthesis of contemplated dinucleotides (i.e., synthesis of one compound at a time). However, and especially where numerous modifications in various positions of a dinucleotide are desired, multiple solid phase synthesis and/or combinatorial library synthesis may advantageously be utilized. Furthermore, and particularly where synthesis of contemplated dinucleotides involves coupling of previously prepared mononucleotides, it is generally contemplated that all known manners of coupling one nucleoside to another nucleoside to form a dinucleotide are deemed suitable.

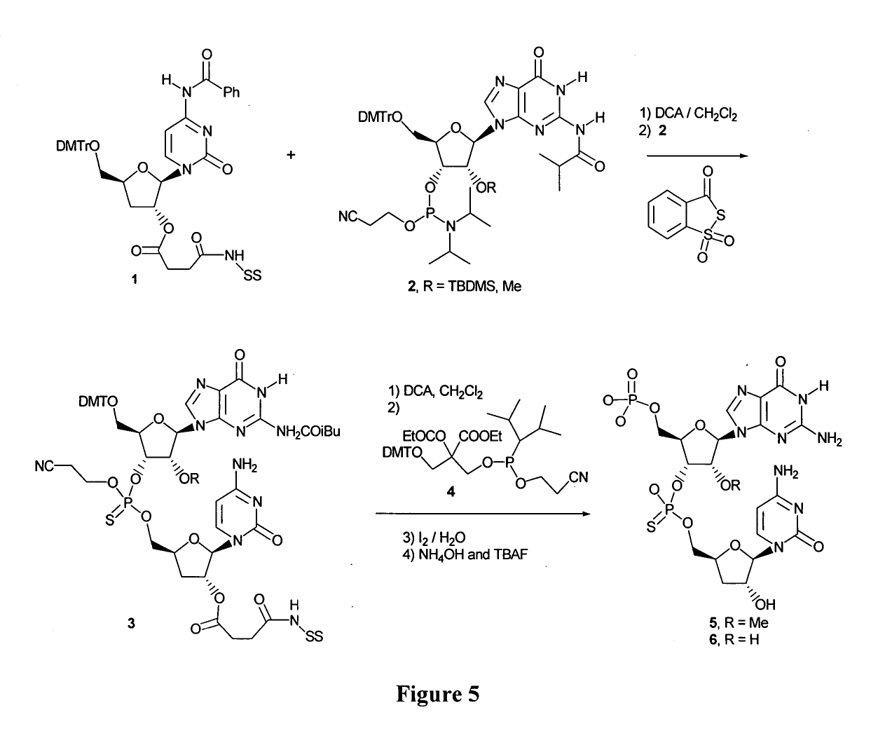

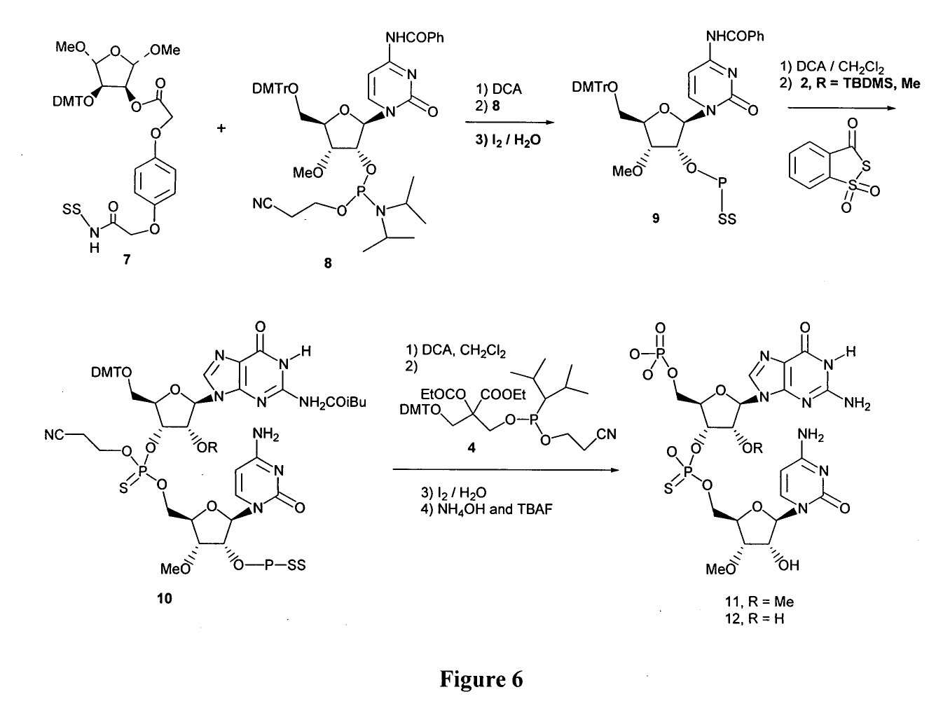

For example, FIG. 5 provides an exemplary synthetic strategy for the preparation of a dinucleotide compound in which the 5′-nucleoside includes a purine base, in which the 3′-nucleoside includes a pyrimidine base, and in which the two nucleosides are coupled together via a phosphorothioate group. Here, an appropriately protected guanosine 3′-O-phosphoramidite is reacted with a 5′-DMT-protected-N4-benzoyl-protected cytidine to form the corresponding dinucleotide in which a trivalent phosphorous atom couples the two nucleosides. Subsequent oxidation will then yield the phosphorothioate linkage, and deprotection results in the dinucleotides 5 and 6. Similarly, as depicted in FIG. 6 below, the suitably protected 3′-nucleotide may be coupled to a solid phase and then reacted with the 5′-nucleotide under conditions substantially similar to those described above.

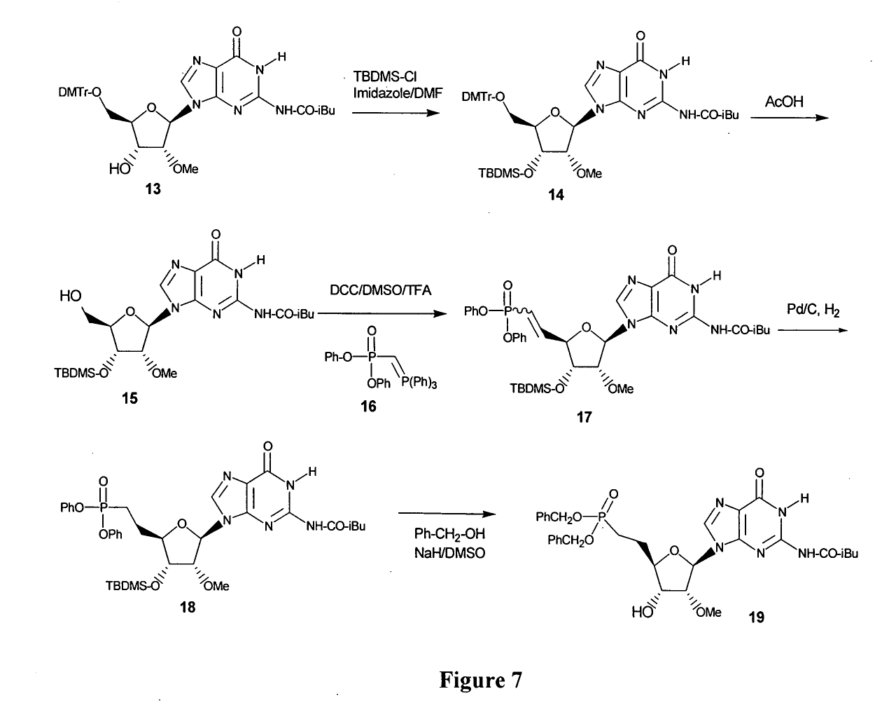

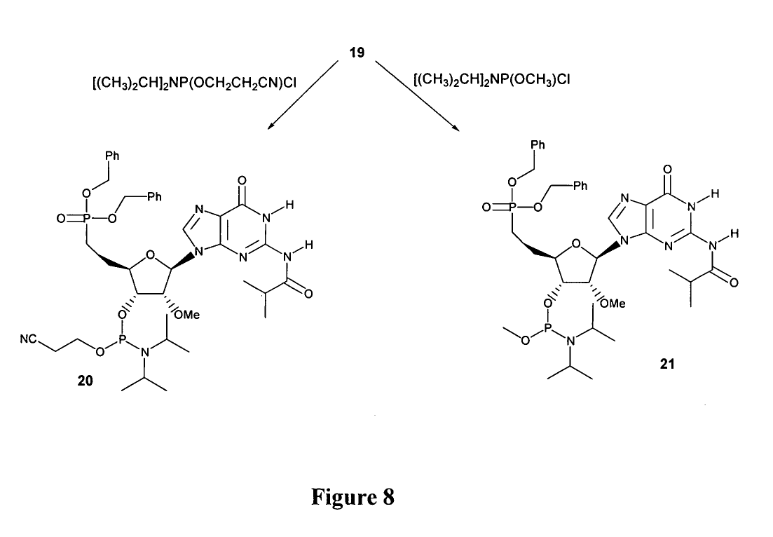

Preparation of exemplary phosphonate nucleotides (here: having a purine base as a heterocyclic base) is depicted in FIG. 7, in which a suitably protected guanosine is reacted to the corresponding phosphonate nucleotide via reaction with a phosphonate and catalytic reduction. FIG. 8 illustrates an exemplary synthetic route that converts the exemplary phosphonate nucleotides of FIG. 7 into a building block for synthesis of contemplated dinucleotide compounds.

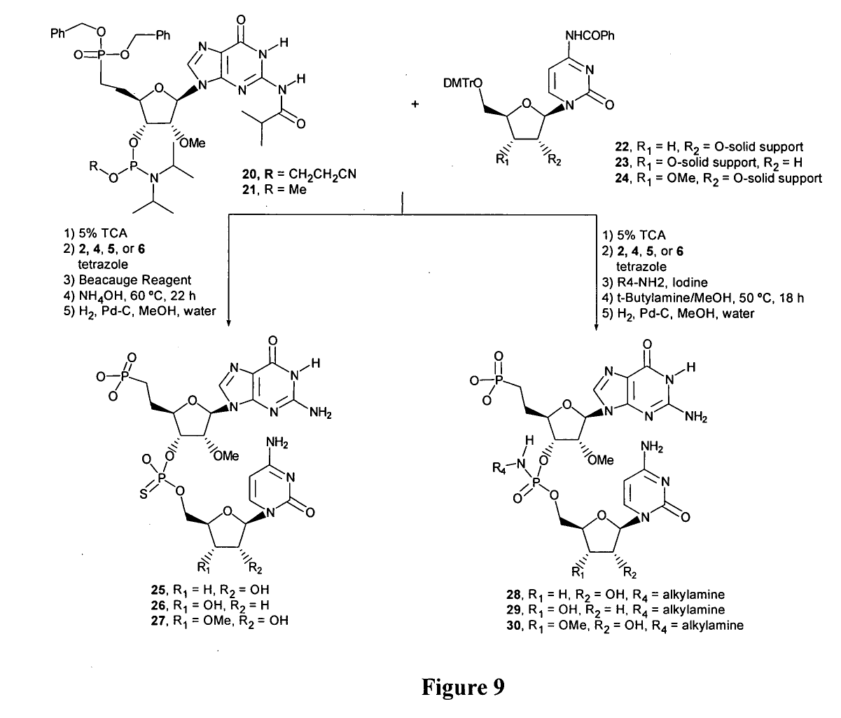

FIG. 9 depicts an exemplary synthetic route for various contemplated dinucleotide compounds in which an exemplary 5′-methylenephosphate dinucleotide phosphorothioate and an exemplary 5′-methylenephosphate dinucleotide phosphoramidate are prepared from a suitably protected purine-nucleotide phosphonate building block and a suitable protected pyrimidine nucleotide. After formation of the dinucleotide, the trivalent phosphorous atom is then further reacted to the corresponding phosphorothioate and phosphoramidate.

Of course, it should be recognized that the nature of the first and second nucleotides may vary considerably without departing from the inventive concept presented herein. For example, the 5′-nucleotide may be prepared as a nucleoside (i.e., without a 5′-phosphate group), and may include a purine or pyrimidine base (which may be substituted to yield a naturally occurring heterocyclic base or a non-naturally occurring heterocyclic base). Furthermore the purine or pyrimidine base may include a CH group in place of an N-atom to yield the corresponding deazanucleotide or dezaznucleoside. Similarly, the sugar moieties may be modified to include substituents that replace the H or OH groups at one or more of the C2′-, C3′-, C4′-, and C5′-atoms. Suitable substituents may provide or remove hydrogen bond donor or acceptor groups, increase or decrease the hydrophilicity, add or remove steric hindrance in the sugar, or force the sugar (or heterocyclic base) in a particularly desirable configuration.

With respect to the coupling of the first and second nucleoside/nucleotide, it should be recognized that all known couplings are deemed suitable and all of the manners of coupling may be employed for use herein. Therefore, contemplated couplings include phosphate groups (and their modifications, e.g., phosphonate, phosphorothioate, boranophosphate, etc.) as well as all other non-phosphate groups with a first reactive group that can form a covalent bond with the first nucleoside/nucleotide, and a second reactive group that can form a covalent bond with the second nucleoside/nucleotide.

Uses of Contemplated Dinucleotide Compounds

It is generally expected that the contemplated compounds have numerous biological activities, and especially contemplated biological activities include in vitro and in vivo inhibition of DNA and/or RNA polymerases, reverse transcriptases, and ligases. While not wishing to be bound by a particular theory or hypothesis, the inventors contemplate that the compounds according to the inventive subject matter act as inhibitors of a viral polymerase, and especially of a viral de novo RNA dependent RNA polymerase (e.g., from HCV). Therefore, contemplated dinucleotides will exhibit particular usefulness as in vitro and/or in vivo antiviral agents (especially against HCV), antineoplastic agents, or immunomodulatory agents. Still further, it is contemplated that dinucleotides according to the inventive subject matter may be incorporated into oligo- or polynucleotides, which will then exhibit altered hybridization characteristics with single or double stranded DNA in vitro and in vivo.

Particularly contemplated antiviral activities include at least partial reduction of viral titers of respiratory syncytial virus (RSV), hepatitis B virus (HBV), hepatitis C virus (HCV), herpes simplex type 1 and 2, herpes genitalis, herpes keratitis, herpes encephalitis, herpes zoster, human immunodeficiency virus (HIV), influenza A virus, Hanta virus (hemorrhagic fever), human papilloma virus (HPV), and measles virus. Especially contemplated immunomodulatory activity includes at least partial reduction of clinical symptoms and signs in arthritis, psoriasis, inflammatory bowel disease, juvenile diabetes, lupus, multiple sclerosis, gout and gouty arthritis, rheumatoid arthritis, rejection of transplantation, giant cell arteritis, allergy and asthma, but also modulation of some portion of a mammal's immune system, and especially modulation of cytokine profiles of Type 1 and Type 2. Where modulation of Type 1 and Type 2 cytokines occurs, it is contemplated that the modulation may include suppression of both Type 1 and Type 2, suppression of Type 1 and stimulation of Type 2, or suppression of Type 2 and stimulation of Type 1.

Where contemplated nucleosides are administered in a pharmacological composition, it is contemplated that suitable dinucleotides can be formulated in a mixture with a pharmaceutically acceptable carrier. For example, contemplated dinucleotides can be administered orally as pharmacologically acceptable salts, or intravenously in a physiological saline solution (e.g., buffered to a pH of about 7.2 to 7.5). Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated nucleosides may be modified to render them more soluble in water or other vehicle, which for example, may be easily accomplished by minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

In certain pharmaceutical dosage forms, prodrug forms of contemplated dinucleotides may be formed for various purposes, including reduction of toxicity, increasing the organ or target cell specificity, etc. Among various prodrug forms, acylated (acetylated or other) derivatives, pyridine esters and various salt forms of the present compounds are preferred. One of ordinary skill in the art will recognize how to readily modify the present compounds to prodrug forms to facilitate delivery of active compounds to a target site within the host organism or patient. One of ordinary skill in the art will also take advantage of favorable pharmacokinetic parameters of the pro-drug forms, where applicable, in delivering the present compounds to a targeted site within the host organism or patient to maximize the intended effect of the compound.

In addition, contemplated compounds may be administered alone or in combination with other agents for the treatment of various diseases or conditions. Combination therapies according to the present invention comprise the administration of at least one compound of the present invention or a functional derivative thereof and at least one other pharmaceutically active ingredient (e.g., antiviral agent, interferon, immunomodulator, etc.). The active ingredient(s) and pharmaceutically active agents may be administered separately or together and when administered separately this may occur simultaneously or separately in any order. The amounts of the active ingredient(s) and pharmaceutically active agent(s) and the relative timings of administration will be selected in order to achieve the desired combined therapeutic effect.

### EXAMPLES

### Synthesis of Selected Phosphorothioate Dinucleotides (FIGS. 5 and 6)

The appropriate cytidine nucleoside (10 μmol) having a 5′-hydroxy function group protected with a dimethoxytriryl (DMT) group and N4 protected with a benzoyl group was derivatised on the control pore glass. The reaction mixture was then treated with 3% dichloroacetic acid to remove the DMT protecting group at 5′-position. The 5′-OH group on solid support was reacted with the appropriately protected Guanosine 3′-O-phosphoramidite 2 with 5′-O-DMTr moiety in presence of tetrazole as a coupling agent. The resulting dinucleotide containing trivalent phosphorus linkage was oxidized with Beaucage reagent to give the dinucleotide 3 with pentavalent phosphorothioate linkage. The 5′-O-DMTr protection of this dinucleotide was removed by mild acid treatment and then further coupled with a commercially available terminal phosphorylating reagent 4. The resulted dinucleotide was then deprotected and cleaved from solid support using aqueous ammonium hydroxide and further purified by HPLC using a reverse phase column providing the desired dinucleotide products 5 and 6.

The dinucleotides 11 and 12 (FIG. 6) were synthesized by similar procedures using solid support 7 with an appropriate linker as the starting material. Cleavage of dinucleotide 11 and 12 from solid support requires NH₄OH treatment at 80° C. for 12 hours, while deprotection of 2′-TBDMS involves TBAF treatment.

### Synthesis of Exemplary Phosphonate Nucleotides (FIGS. 7 and 8)

t-Butyldimethylsilyl chloride (2.06 gm, 13.7 mmol) and imidazole (1.86 gm, 27.4 mmol) were added to a solution of 13 (6.4 g, 9.5 mmol) in 40 ml of DMF. The reaction mixture was stirred at room temperature overnight, then concentrated and dissolved in ethyl acetate. The solution was washed with aqueous sodium bicarbonate solution, water and brine. The organic layer was dried and concentrated. The residue was purified by flash chromatography on a silica gel column using a CHCl₃/MeOH compound eluted with 2% MeOH in CHCl₃ providing 4.95 g (85%) of product 14 as a white foam: silica gel TLC R_{f}0.40 (Hexanes-ethyl acetate, 1/2). ¹H NMR (CDCl₃) δ-0.04 (s, 3H), 0.03 (s, 3H), 0.85 (s, 9H), 0.83 (d, 3H, J=6.8 Hz), 0.96 (d, 3H, J=6.84 Hz), 1.76-1.95 (m, 1H), 3.06-3.18 (m, 1H), 3.38 (s, 3H), 3.48-3.58 (m, 1H), 3.78 (s, 6H), 4.08-4.18 (m, 1H), 4.20-4.38 (m, 2H), 5.87 (d, 1H, J=6.2 Hz), 6.78-6.90 (m, 4H), 7.20-7.60 (m, 9H), 7.85 (s, 1H), 7.94 (b, 1H), 12.00 (b, 1H).

A solution of compound 14 (100 mg, 0.12 mmol) in 10 ml of 80% aqueous acetic acid solution was stirred at room temperature for 2 hours, concentrated and dissolved in ethyl acetate. The solution was washed with aqueous sodium bicarbonate, water and brine. The organic phase was dried and concentrated. The residue was purified by flash chromatography on a silica gel column using 1/0 and 15/1 ethyl acetate-methanol as eluents providing 55 mg (96%) of product 15 as a white foam: silica gel TLC R_{f}0.47 (ethyl acetate-methanol, 15/1). ¹H NMR (CDCl₃) δ 0.00-0.10 (m, 6H), 0.90 (s, 9H), 1.21 (d, 3H, J=4.4 Hz), 1.24 (d, 3H, J=4.4 Hz), 3.26 (s, 3H), 2.66-2.83 (m, 1H), 3.62-3.80 (m, 1H), 3.95-4.02 (m, 1H), 4.05-4.16 (m, 1H), 4.19-4.29 (m, 1H), 4.40-4.50 (m, 1H), 5.11-5.32 (s, 1H, OH), 5.81 (d, 1H, J=6.2 Hz), 7.95 (s, 1H), 9.33 (s, 1H, NH), 12.20 (s, 1H, NH).

Trifluoroacetic acid (78 μl) was added to a stirred solution of 15 (700 mg, 1.45 mmol) and DCC (1.26 g, 6.1 mmol, 4 equiv) in DMSO (7.8 ml) and pyridine (164 μl). The reaction mixture was stirred at room temperature for 24 hours. A mixture of phosphonate 16 (3.19 mmol, 2.2 equiv) and pyridine (300 μl) in DMSO was added. The resulted mixture was stirred at room temperature for 30 hours (monitored by TLC) and then diluted with chloroform upon reaction completion. The solution was washed with water, dried and concentrated. The residue was triturated with chloroform and filtered to remove the solid urea. The crude product was purified by flash chromatography on a silica gel column using Hexanes-chloroform (40:60), chloroform and chloroform:MeOH (98:2) as eluents providing 0.8 g (71%) of product 17 as white foam. ¹H NMR (CDCl₃) δ 0.10 (m, 6H), 0.95 (s, 9H), 1.00 (d, 3H), 1.15 (d, 3H), 2.70 (m, 1H), 3.20 (s, 3H), 4.10 (d, 1H), 4.50-4.70 (m, 2H), 5.80 (d, 1H), 7.00-7.80 (m, aromatic), 10.40 (s, 1H), 12.4 (s, 1H).

A solution of 17 (1.52 g, 1.95 mmol) in 50 ml of methanol and 0.5 ml of acetic acid was stirred over 10% Pd/C (800 mg) under 1 atmosphere of hydrogen for 24 hours. The reaction mixture was filtered through a pat of Celite and washed with methanol. The filtrate was concentrated and purified by flash chromatography on a silica gel column using Hexanes-chloroform (40:60), chloroform and chloroform:MeOH (98:2) as eluents providing 1.16 g (76%) of product 18 as a white foam. ¹H NMR (CDCl₃) δ 0.10 (m, 6H), 0.95 (m, 12H), 1.20 (d, 3H), 2.00 (m, 2H), 3.20 (s, 3H), 4.05 (d, 1H), 4.2 (d, 1H), 4.45 (m, 1H), 5.80 (d, 1H), 6.90 (d, 2H), 7.20-7.80 (m, aromatic), 10.60(s, 1H), 12.30 (s, 1H).

Sodium hydride (400 mg, 60% in oil) was washed with hexanes and reacted with benzyl alcohol (6 ml) in DMSO (8 ml). A solution of 18 (1.16 g, 1.48 mmol) in 15 ml of DMSO was added. The reaction mixture was stirred at room temperature for 4 hours and diluted with ethyl acetate. The solution was washed with aqueous ammonium chloride solution and brine. The organic phase was dried and concentrated. The residue was purified by flash chromatography on a silica gel column Hexanes-chloroform (40:60), chloroform and chloroform:MeOH (98:5) as eluents providing 649 mg (70%) of product 19 as a white foam. ¹H NMR (CD₃OD) δ 1.20 (m, 6H), 2.00 (m, 4H), 2.80 (m, 1H), 3.42 (s, 3H), 3.95 (m, 1H), 4.23 (m, 2H), 5.01 (m, 4H), 5.91 (d, 1H), 7.29 (d, 10H), 8.02 (s, 1H). ³¹P NMR (CD₃OD) δ 35.15.

The so prepared compound may then be reacted with phosphonylating agents to yield exemplary compounds 20 or 21 as depicted in FIG. 8. Here, the phosphonylating reagent [(CH₃)₂CH]₂NP(OCH₂CH₂CN)Cl (0.22 ml, 0.99 mmol) and diisopropylethylamine (0.52 ml, 3.0 mmol) were added to a solution of 19 (0.62 mg, 0.99 mmol) in CH₂Cl₂. The reaction mixture was stirred at room temperature for 4 hours. The reaction mixture was diluted with water, washed with water, and dried over Na₂SO₄. This resulting 20 was used for the next step without further purification. Similarly, the phosponylating reagent [(CH₃)₂CH]₂NP(OCH₃)Cl (0.25 ml, 1.31 mmol) and diisopropylethylamine (0.69 ml, 4.0 mmol) were added to a solution of 19 (0.82 mg, 1.31 mmol) in CH₂Cl₂. The reaction mixture was stirred at room temperature for 4 hours. The reaction mixture was diluted with water, washed with water, and dried over Na₂SO₄. This resulting 21 was used for the next step without further purification.

### Synthesis of Exemplary 5′-Methylenephosphate Dinucleotide Phosphorothioates (FIG. 9)

10 μmol of nucleoside 22, 23 or 24, covalently linked to a solid support (long chain alkyl amine control pore glass) through ester linkage, was reacted with 50 μmol of 20 in acetonitrile containing tetrazole. The reaction mixture was kept for 45 minutes and then washed with CH₃CN followed by CH₂Cl₂. The intermediate was then oxidized to phosphorothioate using Beacauge reagent in CH₃CN. The resin was washed with CH₃CN, dried, and then cleaved from solid support by ammonium hydroxide. The benzyl groups from terminal methylenephosphate were then removed by hydrogenation over Pd/C to give the desired 5′-methylenephosphate dinucleoside phosphorothioate 25, 26 or 27.

### Synthesis of Exemplary 5′-Methylenephosphate Dinucleotide Phosphoramidate (Scheme 5)

10 μmol of nucleoside 22, 23 or 24, covalently linked to a solid support (long chain alkylamine control pore glass) through ester linkage, was treated with 50 μmol of 21 in acetonitrile containing tetrazole. The reaction mixture was kept for 45 minutes and then the resin was washed with CH₃CN followed by CH₂Cl₂. The resulted intermediate was then oxidized to phosphoramidate using Iodine and alkylamine in THF. Then the resin was washed with CH₃CN, dried, and then cleaved from solid support by t-butylamine-MeOH. The benzyl groups from the terminal methylenephosphate were then removed by hydrogenation over Pd/C to give 5′-methylenephosphate dinucleoside phosphoramidate 28, 29 and 30.

### Biological Tests Demonstrating Inhibition of De Novo RNA Synthesis

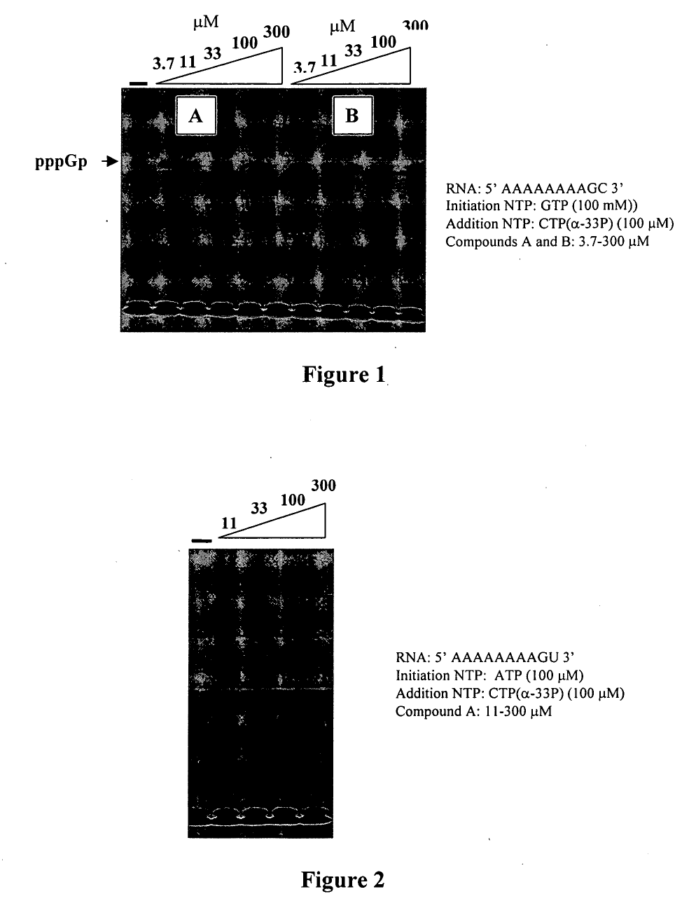

In vitro RNA-dependent RNA polymerase assay was used to evaluate activity of dinucleotide compounds as inhibitors of HCV polymerase. Examples of such compounds are illustrated below as compounds A and B in. In a standard HCV NS5B de novo initiation assay in which a synthetic RNA oligonucleotide (5′ AAAAAAAAAGC 3′) was used as a template and GTP as the initiation nucleotide, compounds A and B showed inhibitory activity against an HCV polymerase with an IC50 of 20 μM and 65 μM, respectively, as depicted in FIG. 1. The high concentration of the initiating nucleotide GTP (100 μM), suggests that the dinucleotide compounds can efficiently compete with GTP for binding to NS5B and thus inhibit the initiation of RNA synthesis.

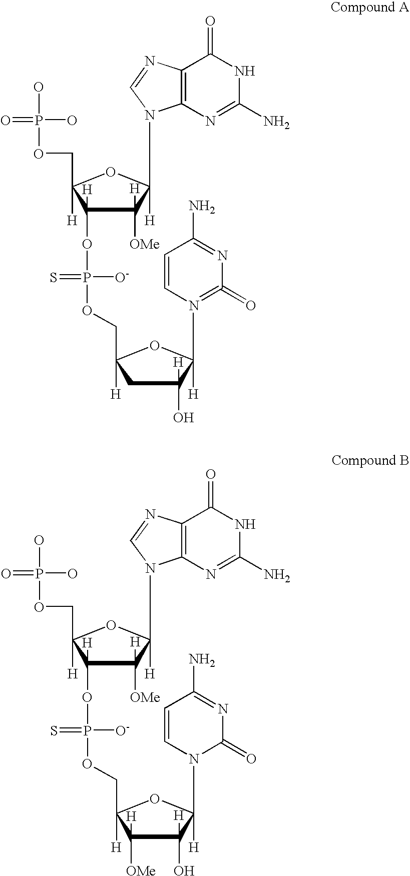

To further determine whether this inhibitory effect of HCV polymerase is specific for a particular initiation nucleotide, de novo initiation assay was performed using ATP as the initiation nucleotide (template RNA: 5′ AAAAAAAAGU 3′). As can be clearly seen in FIG. 2, compound A was still able to inhibit polymerase activity, though less efficiently with an IC50 of ˜90 μM, suggesting that base pairing capability between the dinucleotide compounds and the terminal bases of the template RNA is important for efficient inhibition.

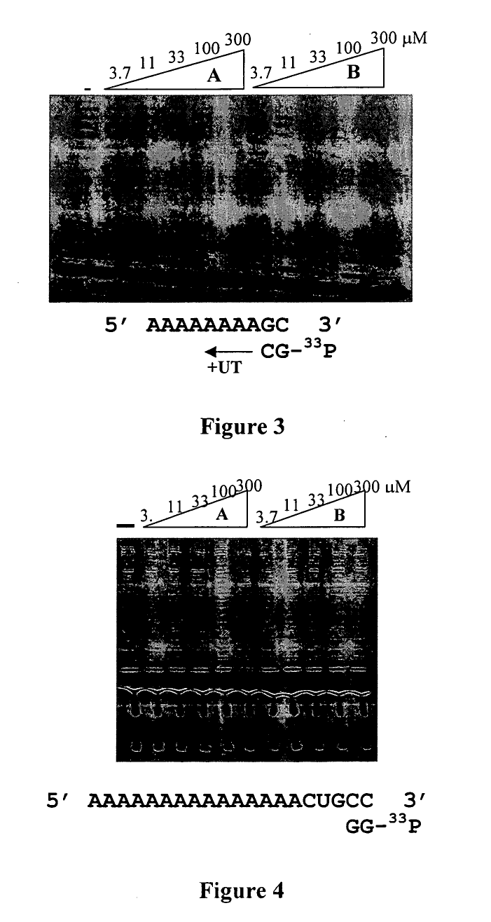

It was also observed that certain dinucleotide analogues were able to inhibit dinucleotide-primed RNA synthesis. As shown in FIG. 3, an end-labeled dinucleotide GpC was used to initiate RNA synthesis (template: 5′ AAAAAAAAGC 3′), compound A inhibited formation of elongated products with an IC50 of 50 μM, while compound B showed a similar activity (IC50˜80 μM). Furthermore, this inhibitory activity is sequence specific. When a different dinucleotide primer (GpG) was used to prime RNA synthesis from the. RNA template 5′ AAAAAAAACC 3′, both compound A and B (analogues of GpC) lost the ability to inhibit the polymerase activity as depicted in FIG. 4. These observations further support the model that base pairing capability between the dinucleotide compound and the template terminal bases is important for achieving sufficient inhibition of HCV polymerase.

Thus, specific embodiments and applications of dinucleotide compounds have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A compound comprising a dinucleotide of the structure A-B, wherein the dinucleotide inhibits synthesis of a polymerase that initiates RNA replication de novo, and wherein A and B independently comprise a nucleoside, a nucleoside analog, a nucleotide, or a nucleotide analog.

2. The compound of claim 1 wherein A comprises a purine nucleoside or purine nucleoside analog.

3. The compound of claim 1 wherein B comprises a pyrimidine nucleoside or pyrimidine nucleoside analog.

4. The compound of claim 1 wherein a sugar moiety of A is covalently bound to B.

5. The compound of claim 1 wherein A is covalently bound to B via a chemical group that includes a phosphorous atom.

6. The compound of claim 5 wherein the chemical group is selected from the group consisting of a phosphate, a phosphorothioate, a phosphonate, a phosphonamide.

7. The compound of claim 1 wherein at least one of A and B includes a modified sugar.

8. The compound of claim 7 wherein the modified sugar includes a 2′-methoxy group or a 3′-methoxy group.

9. The compound of claim 1 wherein at least one of A and B comprises guanosine.

10. The compound of claim 1 wherein at least one of A and B includes a heterocyclic base that forms at least two hydrogen bonds with a terminal heterocyclic base of a template RNA.

11. The compound of claim 1 wherein the de novo initiation of RNA replication is initiated by guanosine triphosphate, adenosine triphosphate, or a dinucleotide.

12. The compound of claim 1 wherein the dinucleotide has a structure according to Formula 1 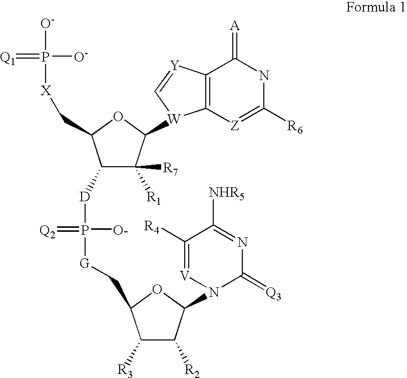
wherein Q₁, Q₂, and Q₃ are independently O or S;
X is O, S, NH, NR, CH₂, CF₂, CHR, or a bond between the C5′-carbon and the P atom;
Y is CH, COR′, or N, wherein R′ is R, CN, COOH, COOR, CONHR, or C(═NH)NH₂;
V and Z are independently N, CH, or CR;
W is N or C;
A is O, S, NH, or NR;
D is O, S, NH, or CH₂;
G is O, S, NH, CH₂, CF₂, or a bond between the C5′-carbon and the P atom;
R₁, R₂, R₃ are independently H, OH, OR, R, halo, CF₃, CCl₃, CHCl₂, CH₂OH, NO₂, CN, N₃, SH, SR, NH₂, NHR, NHCOR, NHSO₂R, NHCONHR, NHCSNHR;
R₄ is R, halogen, haloalkyl, haloalkenyl, or substituted heteroaryl;
R₅ is H, NH₂, NHR, NR₂, NHCOR, NHSO₂R, heterocycle, COR, or SO₂R;
R₆ is NH₂, NHCOR, NHSO₂R, NHNH₂, NHNHR, NHR, or NR₂;
R₇ is H, OH, SH, OR, SR, R, halogen, CF₃, CN, CHCl₂, CH₂OH, N₃, NH₂, or CH₂Cl; and
wherein R is alkyl, alkenyl, alkynyl, aryl, or alkaryl.

13. The compound of claim 1 wherein the dinucleotide has a structure according to Formula 2 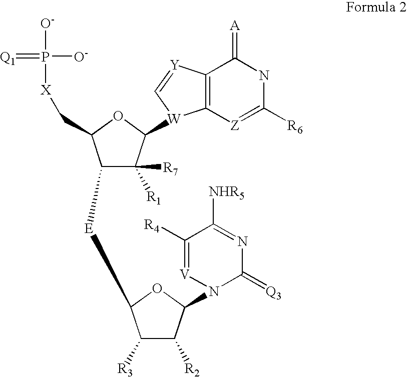
wherein E is —O—P(Q₂)(NR₈R₉)—O—, —NHC(O)(CH₂)₁₋₁₀C(O)—, —NH-Heterocycle-O—, —O(CH₂)₁₋₁₀C(O)—, or —O-Heterocycle-O—;
wherein Q₁, Q₂, and Q₃ are independently O or S;
X is O, S, NH, NR, CH₂, CF₂, CHR, or a bond between the C5′-carbon and the P atom;
Y is CH, COR′, or N, wherein R′ is R, CN, COOH, COOR, CONHR, or C(═NH)NH₂;
V and Z are independently N, CH, or CR;
W is N or C;
A is O, S, NH, or NR;
R₁, R₂, R₃ are independently H, OH, OR, R, halo, CF₃, CCl₃, CHCl₂, CH₂OH, NO₂, CN, N₃, SH, SR, NH₂, NHR, NHCOR, NHSO₂R, NHCONHR, NHCSNHR;
R₄ is R, halogen, haloalkyl, haloalkenyl, or substituted heteroaryl;
R₅ is H, NH₂, NHR, NR₂, NHCOR, NHSO₂R, heterocycle, COR, or SO₂R;
R₆ is NH₂, NHCOR, NHSO₂R, NHNH₂, NHNHR, NHR, or NR₂;
R₇ is H, OH, SH, OR, SR, R, halogen, CF₃, CN, CHCl₂, CH₂OH, N₃, NH₂, or CH₂Cl;
R₈ and R₉ are independently H, (CH₂)₁₋₁₀—NH₂, (CH₂)₁₋₁₀—C(═NR)NHR, (CH₂)₁₋₁₀—NH—C(═NR)NHR, (CH₂)₁₋₁₀—COOR, (CH₂)₁₋₁₀—CONHR, (CH₂)₁₋₁₀-Heterocycles, or R; and
wherein R is alkyl, alkenyl, alkynyl, aryl, or alkaryl.

14. The compound of claim 1 wherein the dinucleotide has a structure according to Formula 3 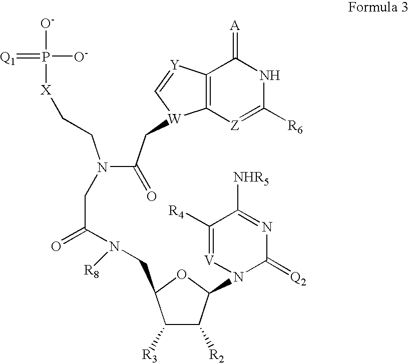
wherein X is O, S, NH, NR, CH₂, a covalent bond between the P atom and the CH₂ group, CF₂, or CHR;
Q₁ and Q₂ are independently O or S;
Y is CH, COR′, or N, wherein R′ is CN, Me, COOH, COOR, CONHR, C(═NH)NH₂, or R;
Z and V are independently N, CH, or CR;
W is N or C;
A is O, S, NH, or NR;
R₂ and R₃ are independently H, OH, OR, R, halo, CF₃, CCl₃, CHCl₂, CH₂OH, NO₂, CN, N₃, SH, SR, NH₂, NHR, NHCOR, NHSO₂R, NHCONHR, NHCSNHR;
R₄ is R, halogen, haloalkyl, haloalkenyl, or substituted heteroaryl;
R₅ is H, NH₂, NHR, NR₂, NHCOR, NHSO₂R, heterocycle, COR, or SO₂R;
R₆ is NH₂, NHCOR, NHSO₂R, NHNH₂, NHNHR, NHR, or NR₂;
R₈ is H or R; and
wherein R is alkyl, alkenyl, alkynyl, aryl, or alkaryl.
